(19) United States
(12) Patent Application Publication
(10) Pub. No.: US 2009/0163893 A1
(43) Pub. Date: Jun. 25, 2009

(54) SURGICAL DRAINS

(75) Inventors: John C. Opie, Scottsdale, AZ (US); Stephen J. Joyce, Phoenix, AZ (US); Thomas Izdebski, Phoenix, AZ (US)

Correspondence Address: FENNEMORE CRAIG, 3003 NORTH CENTRAL AVENUE, SUITE 2600, PHOENIX, AZ, 85012, US

(73) Assignee: JS Vascular, Inc., Scottsdale, AZ (US)

(21) Appl. No.: 12/343,231

(22) Filed: Dec. 23, 2008

Related U.S. Application Data
(63) Continuation of application No. 10/559,400, filed on May 25, 2006, now abandoned.
(60) Provisional application No. 60/476,663, filed on Jun. 5, 2003.
(60) Provisional application No. 60/539,158, filed on Jan. 26, 2004.

(51) Int. Cl. A61M 27/00

(52) U.S. Cl. 604/541

(57) ABSTRACT

Disclosed are surgical drains for use in surgical wounds. The drains are placed in the wound in the body and draw in unwanted bodily material. The drains can be of any shape, but preferably have a generally circular cross section or include a generally flat top surface, a generally flat bottom-surface and two sides connecting the top surface and bottom surface. The drain has an outer surface, two ends, a length and an interior lumen that may extend substantially along the length. At least one row of openings is formed in the outer surface along the longitudinal axis. The openings in a single row may comprise large cross-sectional openings alternating with small cross-sectional openings. If a plurality of rows are utilized, the small cross-sectional openings may not be included. It is preferred that the large cross-sectional openings in one row be offset, or staggered, from the large cross-sectional openings in a neighboring row. The flat drain may include large openings on one or more of the flat surfaces and smaller openings on the sides. It is preferred, but not required, that at least some of the openings be formed in the base of a channel formed in the outer surface of the drain.

### RELATED APPLICATIONS

This application claims priority to U.S. Provisional Application Ser. No. 60/476,663, filed Jun. 5, 2003, and to U.S. Provisional Application Ser. No. 60/539,158, filed Jan. 26, 2004, the disclosure of which is hereby incorporated by reference.

### FIELD OF THE INVENTION

This invention relates to medical devices and, more specifically, to high-performance surgical drains providing improved drainage.

### BACKGROUND OF THE INVENTION

Modern surgery increasingly attempts to minimize the length and trauma of surgery, and surgical incisions. Today, incisions can be quite small, but despite the small entry and exit incisions, significant surgery, resulting in wounds to internal body tissue, continues to be done inside the human body either percutaneously, laproscopically or in open-cut surgeries. As a consequence, considerable undermining may occur. Current surgical drains are not ideally suited to such circumstances. Most current surgical drains are short in length and in certain circumstances their surface features (particularly the size and number of openings for draining body fluids, fibrin and clots and other particulate wound debris (collectively, “bodily materials”)) may lead to drain occlusions resulting in drain blockage and failure, in which case unwanted bodily materials may remain in the body. Hematomas then form and additional surgeries may be required to drain or repair the area, which to some extent defeats the aims and outcomes of minimally invasive surgical techniques.

Devices that drain surgical incisions typically comprise an implantable, inflow drain section hereafter, “inflow section,” “surgical drain” or “drain”) that is (at least partially) placed into the patient's body where it is in communication with the patient's bodily materials. This drain is usually connected to a low-profile transition component or connector section (which is generally an elongated tube that leads from the inflow section and extends towards the outside of the patient's body), which in turn is connected to an outflow section. The outflow section in turn is connected to a device, such as a vacuum device (like a deformable grenade-shaped container or other suction (or vacuum) device or any type of suitable reservoir), that draws bodily materials into it and receives the bodily materials collected from the drain. Most existing vacuum reservoirs possess a double, one-way valve mechanism that permits filling the reservoir with bodily materials through one of the one-way valves and emptying the reservoir with the other of the one-way valves, but the collected bodily materials cannot back up from the reservoir into the wound.

Typically, present drains available for surgical incisions have either a series of small, parallel perforations running the length of the drain, or a series of narrow, linear crevices. Unwanted bodily materials are drawn (usually via vacuum) into the drain through the perforations or crevices and drawn through the transition component, the outflow component and into the reservoir. The drains are usually of a flattened oval or a circular shape and are usually about 30 cm long.

There are several fairly commonly observed/encountered problems with current surgical drains. In drains that have a series of small parallel perforations, such as a drain known as the oval Jackson-Pratt drain (“J-P”), bodily materials can occlude the relatively small holes in the drain which allows bodily materials to accumulate in the wound rather than being removed via the drain. The entrance holes in this drain are small, and when presented with blood clots, or if buried in subcutaneous fat, the drain tends to clog and fail.

Another problem with perforated drains is the structure and location of the holes themselves. The openings act as weak points in the structure of the drain and are sometimes called “stress risers,” and can break or tear when the drain is pulled during extraction from the wound. If this occurs, an incision may have to be made to remove the drain. Another deficiency with perforated drains is their length. Most current drains were not designed for the long tunnels in wounds created in an appreciable number of current minimally-invasive surgeries. The most convenient way to overcome this deficiency at present is to implant two or more drains into a wound. That is reasonably easy to do, but it is unnecessarily expensive and requires more than one drain to be extracted thereby increasing the patient's discomfort.

Another common style of drain is known as the Blake drain. The Blake drain alleviates at least one of the deficiencies of the J-P drain; it has no “stress risers” that would cause it to tear during normal usage, it has only four longitudinal, narrow grooves into which the body fluids can enter. These grooves, however, are narrow—approximately one millimeter wide. The drain has slightly larger cooperating egress capillary lumens than the J-P drain.

Although the problem of “stress risers” has generally been solved with the Blake drain, this drain's capacity to clog, especially in situations when locally inserted, surgical procoagulants like Surgicel, Avitene, Gelfoam, or Tisseel or other forms of collagen/cellulose powder are used and/or if charred tissue particles secondary to cautery coagulation are present, or just when fibrin, blood clots, and/or fat globules are present, is relatively high. This drain is prone to clog at its narrow ingress grooves or narrow egress channels where the collected bodily materials are transported from the drain to a tube that extends out of the body.

An additional problem with known drains is that they can internally clog. Near one end of a Blake drain its multiple cooperating lumens merge into one central lumen. It is at this spot there is a choke point within the drain. Essentially, the problem is that the cross-sectional area of the central lumen is significantly less than the combined cross-sectional areas of the lumens leading to the central lumen. The exterior tube to which the drain attaches has a lumen of 3 mm while the drains have, in the case of the Blake drain, four rectangular lumens of about 2×2 mm amounting to 12 mm² total cross-sectional area, while the J-P drain has a single oblong-shaped lumen measuring about 1 mm×5 mm whereas the exterior tubing has a 3 mm total circular lumen. This results in a 50% reduction in fluid transfer capacity at this point for the Blake drain. Thus some drains are apt to clog at this choke point. It is common when inspecting removed drains to find the exterior of the drain unclogged, but the drain clogged at the choke point and fluid and/or debris extending back up into the drain.

### SUMMARY OF THE INVENTION

The present invention relates to an improved a surgical drain for draining bodily materials from wounds. The present invention may also possibly be used for supplying medications to wounds, particularly for minimally invasive surgical procedures. Such procedures often have small entrance incisions and maximal undermining, which have a significant potential to form clots. The invention is usable in all non-minimally invasive surgical procedures as well.

The present invention comprises a surgical drain that is preferably longer; has more overall opening area per overall drain surface area to receive bodily materials and/or a larger internal lumen than known drains. The surgical drain of the present invention comprises a single, continuous elongate member (and the single, continuous member could be made from several connected sections) having any or all of the following: (1) the drain itself, (2) a connector tube, (3) an extension segment, (4) an optional trocar capable of passing though the skin, (5) a receiving reservoir connector segment, and (6) a receiving connector of a receiving reservoir, which is preferably a larger volume, grenade style, vacuum bulb or an underwater seal, wall suction unit adapter.

The drain preferably comprises a body of highly pliant, biocompatible, plastic elastomeric silicone (although any suitable material may be used), which has a relatively large interior lumen (which preferably is formed in the center of the drain and preferably runs the length of the drain).

There are two preferred drain embodiments; One preferably has a circular cross-sectional area, although any suitable shape may be utilized. This drain has large cross-sectional area openings and small cross-sectional area openings, whereby in the preferred embodiment, the large cross-sectional area openings are circular (although any suitable shape may be used) and about 4 mm in diameter and the small cross-sectional openings are rectangular (although any suitable shape may be used) and are about 1.3 mm×3.0 mm. In the preferred embodiment, the rectangular openings are positioned at the base of a channel (which may be about 0.5 mm deep) in the outer surface of the drain. The purpose of the channel and/or positioning the openings at least partially in the base of the channel and/or using non-circular openings, such as rectangular or square openings, is that each of these techniques prevents in-growth by body tissue and/or is more difficult for the body to occlude the openings.

Preferably, this embodiment has four channels running the length of the drain, wherein the cross-sectional area of the lumen is preferably, although not necessarily, approximately the same as the combined cross-sectional areas of the channels, and the lumen has a diameter of about 6 mm.

Another of the preferred embodiments is a drain with a generally flat top surface and flat bottom surface and two sides. There are large cross-sectional, circular openings (although any suitable shape may be used) on the top surface and/or bottom surface and smaller rectangular (although any suitable shape may be used) openings on at least one of the sides. This drain preferably has one or more internal ribs that prevent the drain from collapsing when vacuum is applied. The smaller openings are preferably offset, or staggered, from the position of the large openings.

A drain according to the present invention compromises is sufficient strength to withstand at least 3-5 pounds of longitudinal pull and not fracture or separate.

The present drain with large ingress and egress openings can provide adequate drainage of large particles of bodily materials, for example, up to 4-6 mm.

The external section of the drain may have a lumen of 6 mm in diameter to connect to an outflow tube (preferably having the same size lumen) that eventually leads to a reservoir. By including a larger than typical outflow tube, no choke point exists with this high performance drain, either inside the wound or outside the body. Thus the likelihood of a clog forming in the drain is further diminished.

### BRIEF DESCRIPTION OF THE DRAWINGS

FIG. 1 is a planar view of a drain according to the invention.

FIG. 2 is a close-up, partial perspective view of the drain of FIG. 1.

FIG. 3 is a planar view of the drain of FIG. 1 attached to other devices that optionally may be used in a drain system.

FIG. 4 is a cross-sectional view taken along line A-A of FIG. 3.

FIG. 5 is an exploded view of the system of FIG. 3

FIG. 6 is a perspective view of the drain of FIG. 1.

FIG. 7 is a planar perspective view of the drain of FIG. 1.

FIG. 8 is a cross-sectional view taken along line B-B of FIG. 7.

FIG. 9 is the drain of FIG. 1 attached to an extension tube and to a trocar 180.

FIG. 10 is a perspective view of another drain according to the invention.

FIG. 11 is a close-up, partial view of the drain of FIG. 10.

FIG. 12 is a planar view of the drain of FIG. 10 attached to other devices that optionally may be used in a drain system.

FIG. 13 is a cross-sectional view taken along lines C-C of FIG. 12.

FIG. 14 is an exploded view of the system of FIG. 12.

FIG. 15a is a side view of the drain of FIG. 10.

FIG. 15b is an end view of the drain of FIG. 15a.

FIG. 15c is a top view of one embodiment of the drain of FIG. 10.

FIG. 15d is a perspective view of the drain of FIG. 10.

### DETAILED DESCRIPTION OF THE PREFERRED EMBODIMENTS

Reference shall now be made to the accompanying figures, wherein the purpose is to describe preferred embodiments of the invention and not to limit same. A drain according to the invention preferably includes a single, continuous, elongate, flexible member, and any suitable material, size, thickness or shape may be utilized.

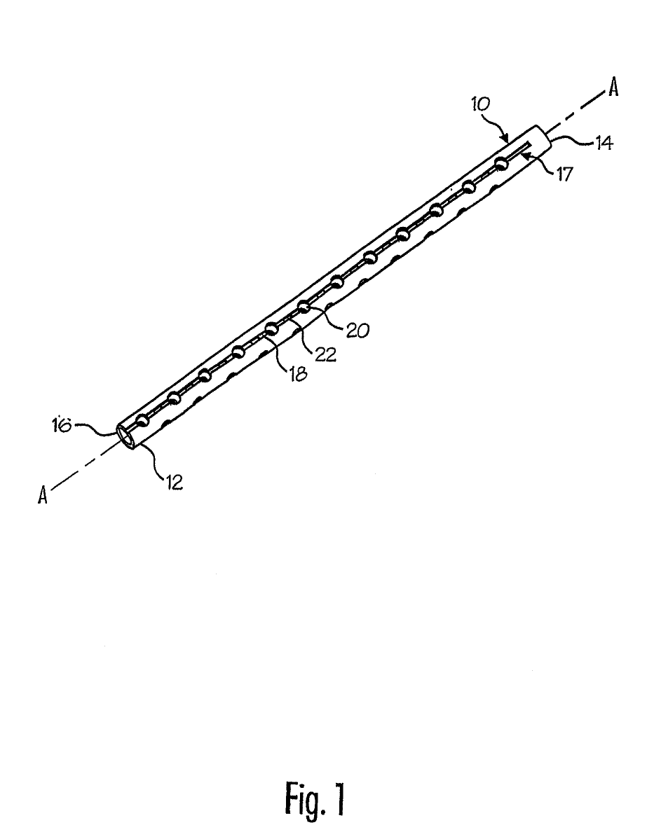

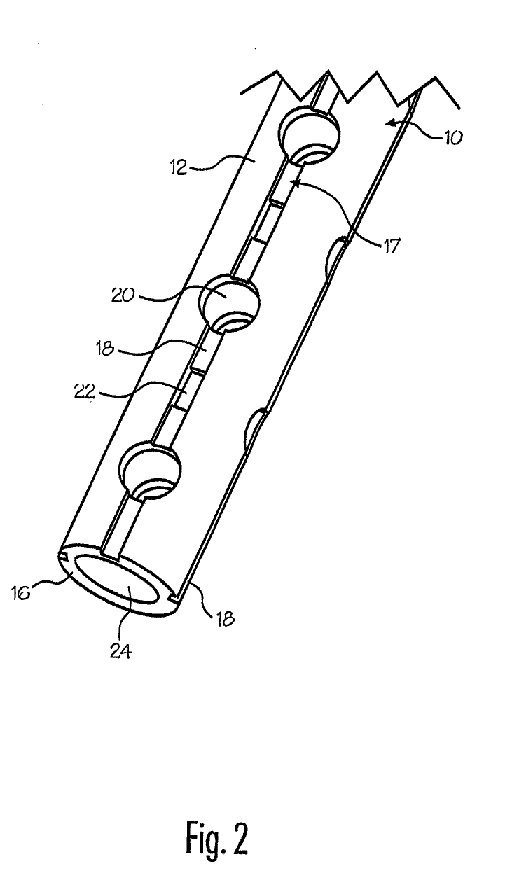

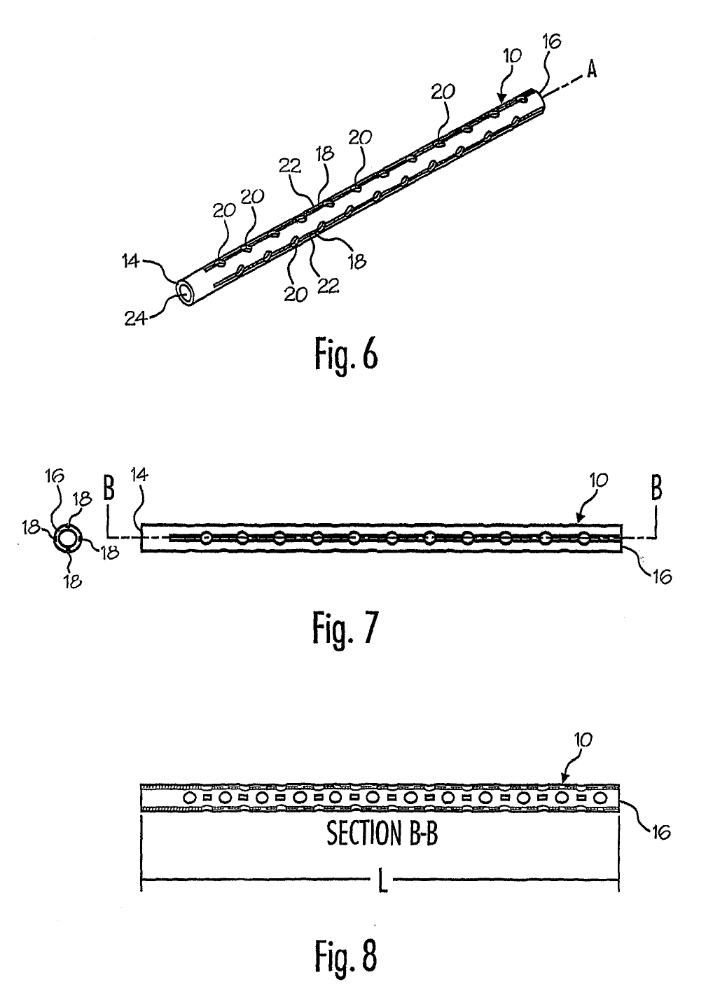

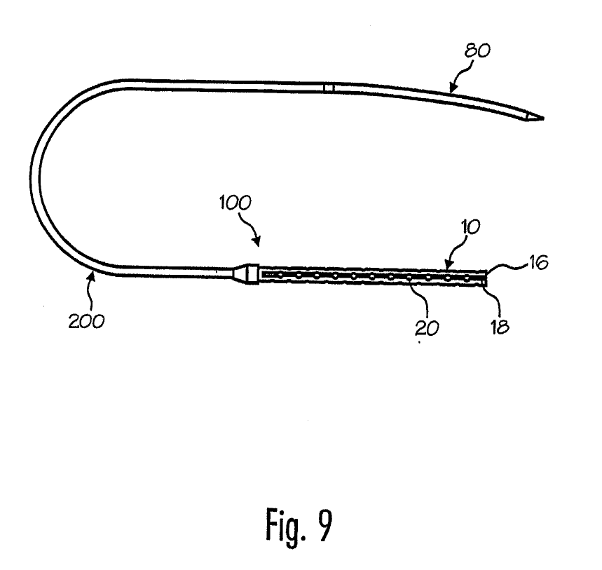

As shown in FIGS. 1-8, a drain 10 and optional drain system 100 utilizing the drain are illustrated. Drain 10 is generally an elongated, tubular member and is preferably injection molded of a flexible plastic or rubber matter, such as silicone rubber. Any suitable method of manufacture or type of material may be used, however. Drain 10 has an exterior surface 12, a first end 14, a second end 16 and an internal lumen 24, preferably having a circular cross-section and a diameter of about 6 mm, extending therethrough. The purpose of lumen 24 is to receive bodily material collected from the outer openings in drain 10 and transport the bodily material out of the body and into a collection reservoir, usually through an outflow tube. Any suitable structure capable of performing this function could be used.

Drain 10 has a longitudinal axis A and a length L. At least one row 17 of openings is formed in outer surface 12. Each row 17 as shown includes an optional channel 18 (which is preferably about 0.5 mm deep) that extends substantially the length L of drain 10. Channel 18 as shown has a rectangular cross section and terminates at end 16. The purpose of channel 18 is to prevent or slow the in-growth of body tissue and prevent openings positioned in, or at least partially in, the channel from being occluded.

A series of openings 20 are formed along the length of row 17. Openings 20 are for collecting bodily materials, are in fluid communication with lumen 24 and transport bodily materials to internal lumen 24. Any openings suitable for this purpose may be used. In this embodiment, openings 20 are circular, equally-spaced along row 17, and have a diameter of about 4 mm.

Row 17 preferably includes optional secondary openings 22. Openings 22 are formed in the base of channel 17, are in fluid communication with lumen 24 and, as shown, one opening 22 is positioned between each opening 20, although openings 22 may be positioned anywhere along row 17, and may be positioned along a separate row, or anywhere on drain 10 as long as the overall structure of the drain is not prone to tearing upon removal. The purpose of openings 22 is to transport additional bodily materials to lumen 24 where they are ultimately removed from the body. As shown, each opening 22 is preferably rectangular and has dimensions of approximately 1.3 mm×3.0 mm, although any suitable size or shape may be used, although non-circular openings, such as rectangular openings, are more difficult for the body to occlude. As shown, openings 22 are entirely positioned in the base of channel 18, but may be only positioned partially in the base.

The preferred embodiment of drain 10 includes a plurality of (and preferably four) rows 17 of alternating openings 20 and 18, wherein rows 17 are equally radially spaced about outer surface 12 so as to increase the accumulative area of opening available to draw bodily materials into lumen 24 and out of the body, while at the same time making the drain less prone to tearing upon removal. It is preferred that each row 17 be positioned in such a manner that the large openings 20 are offset from the large openings 20 in the neighboring row (or juxtaposed row). This tends to maximize the amount of drain material between openings and alleviates weak sections that may tear when the drain is removed.

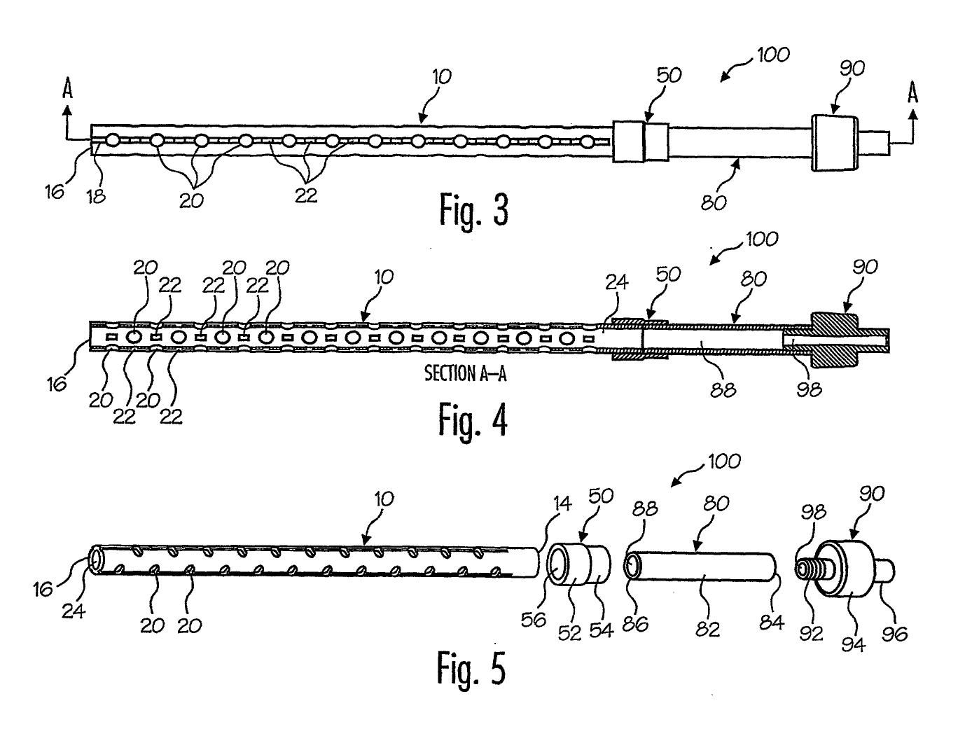

An optional drain system 100 utilizing drain 10 is shown in FIGS. 3-5. The purpose of the system components used with drain 10 is to transport bodily materials to a reservoir (not shown) and any suitable components or system may be used for this purpose. As shown, a collar 50, having a first section 52, a second section 54 and a passage 56, couples drain 10 to an outflow tube 80, which has another surface 82, a first end 84, a second end 86 and an internal passage 88. A connector 90 attaches to outflow to be 80 and has a connector 92, a main body portion 94, a second connector 96 and internal passage 98.

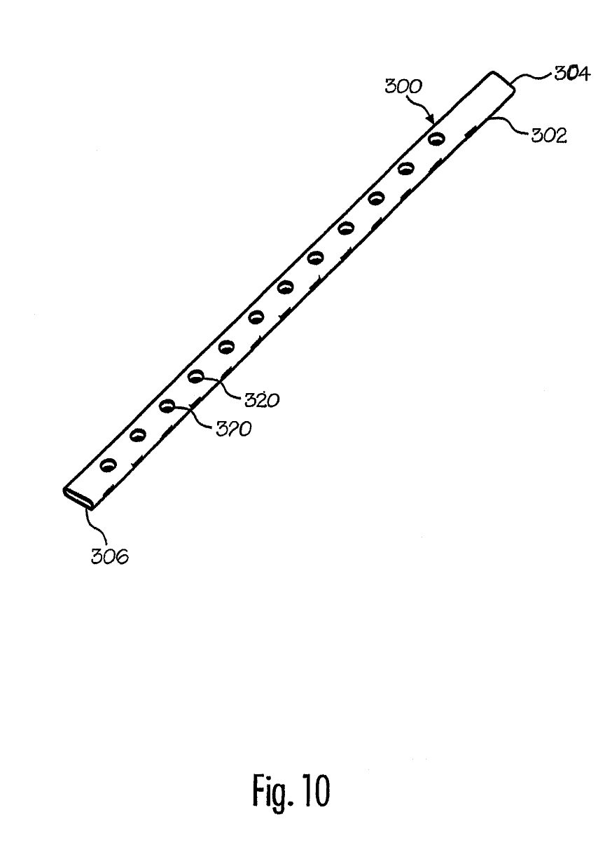

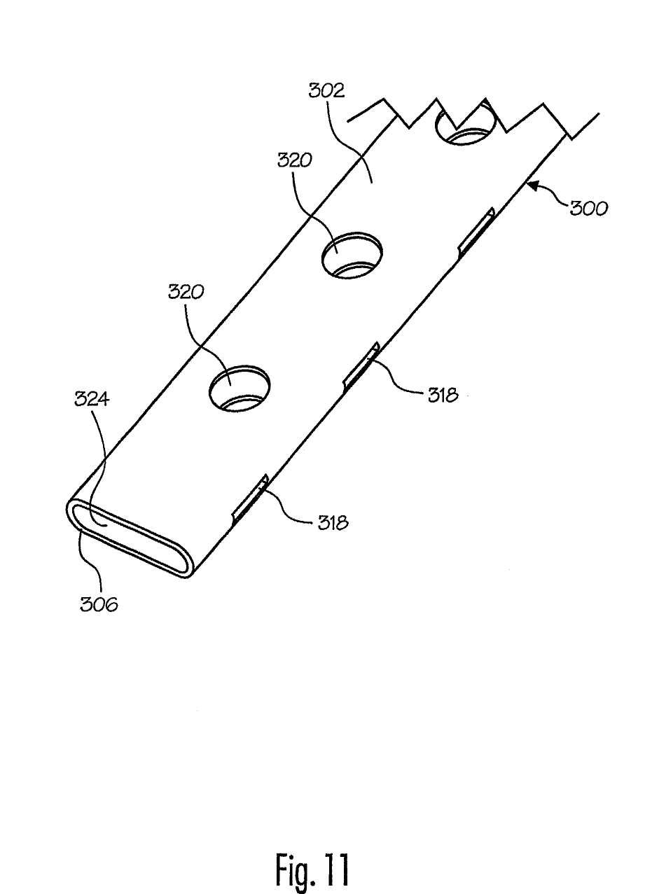

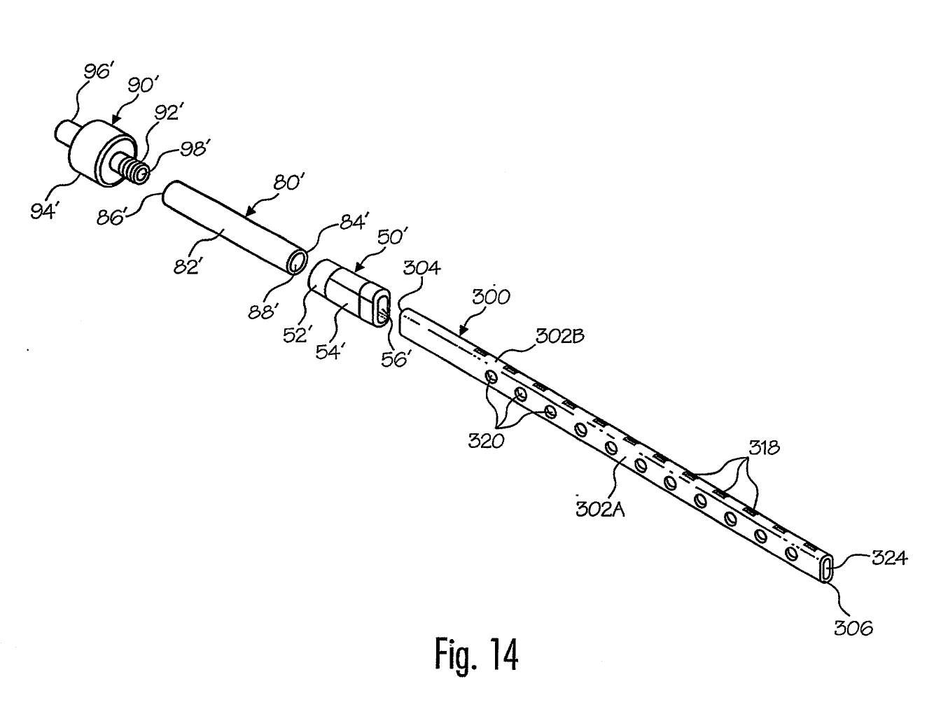

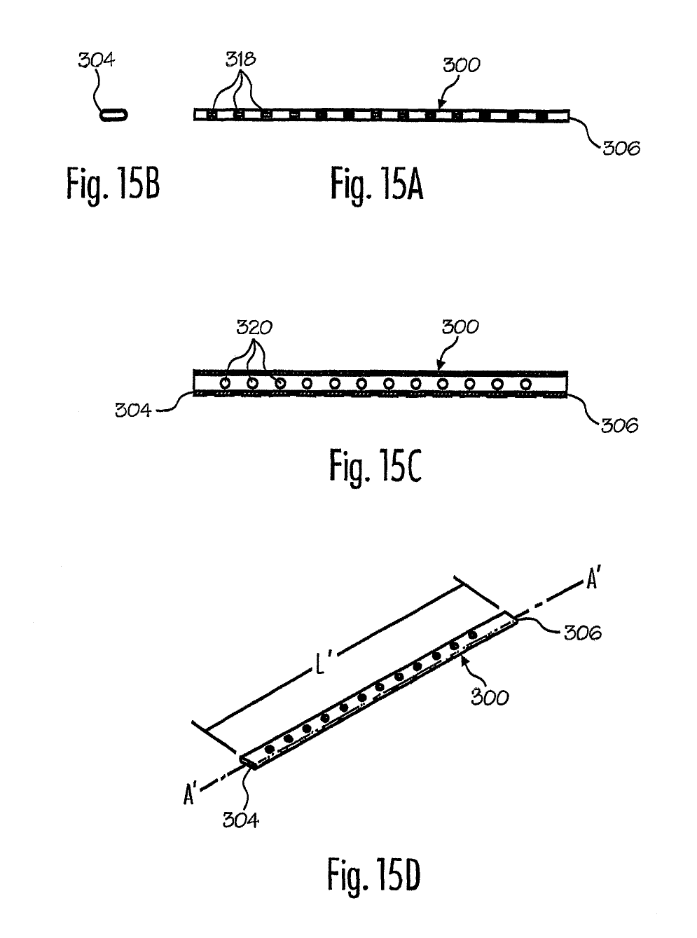

As shown in FIGS. 10-15d, a drain 300 and optional drain system 400 utilizing drain 300 are illustrated. Drain 300 is generally an elongated, tubular member and is preferably injection molded of a flexible plastic or rubber matter, such as silicone rubber. Any suitable method of manufacture or type of material may be used, however. Drain 300 has an exterior surface 302, a first end 304, a second end 306, a generally flat top surface 301, a generally flat bottom surface 303, side surfaces 305, 307 and an internal lumen 324, preferably having a cross-section substantially similar to the cross-sectional shape of drain 300, extending therethrough. The purpose of lumen 324 is to receive bodily material collected from the outer openings in drain 300 and transport the bodily material out of the body and into a collection reservoir. Any suitable structure capable of performing this function could be used, and lumen 324 could have a cross-sectional area of between 20 mm² and 30 mm². One or more ribs (not shown) or other structure are preferably positioned in lumen 324 and keep surface 301 at least partially separated from surface 303 when vacuum is applied to drain 300.

Drain 300 has a longitudinal axis A′ and a length L′. At least one set of openings 320 is formed in either top surface 301 or bottom surface 303, and openings 320 are preferably formed in both top surface 301 and bottom surface 303.

Openings 320 are for collecting bodily materials, are in fluid communication with lumen 324 and transport bodily materials to internal lumen 324. Any openings suitable for this purpose may be used, and particularly openings large enough to ingest particles having one or more dimensions of between 4 mm and 6 mm. In this embodiment, openings 320 are circular, equally-spaced along top surface 301 and/or bottom surface 303 (and openings 320 in surface 301 may be offset from the openings in surface 303), and have a diameter of about 4 mm.

Optional secondary openings 322 are preferably formed in one or both of side surfaces 305, 307, are in fluid communication with lumen 324 and, as shown, each opening 322 is staggered between each opening 320, although openings 322 may be positioned anywhere along sides 305 and/or 307, and may be positioned anywhere on drain 300 as long as the overall structure of drain 300 is not prone to tearing upon normal removal. The purpose of openings 322 is to transport additional bodily materials to lumen 324 where they are ultimately removed from the body. As shown, each opening 322 is preferably rectangular and has dimensions of approximately 2.0 mm×3.0 mm, although any suitable size or shape may be used, although non-circular openings, such as rectangular openings, are more difficult for the body to occlude.

An optional channel(s) (which would be preferably about 0.5 mm deep) could extend partially along length L′ on one or both sides 305, 307 and/or surface 301 and/or surface 303 of drain 300. The channel(s) would preferably have a rectangular cross section and be of substantially the same design as previously-described channel 18. The purpose of the channel(s) would be to prevent or slow the in-growth of body tissue and prevent openings positioned in, or partially in, the channel(s) from being occluded.

The thickness of outer wall 12 or outer wall 302 could be 3.0 mm, although any suitable thickness may be used.

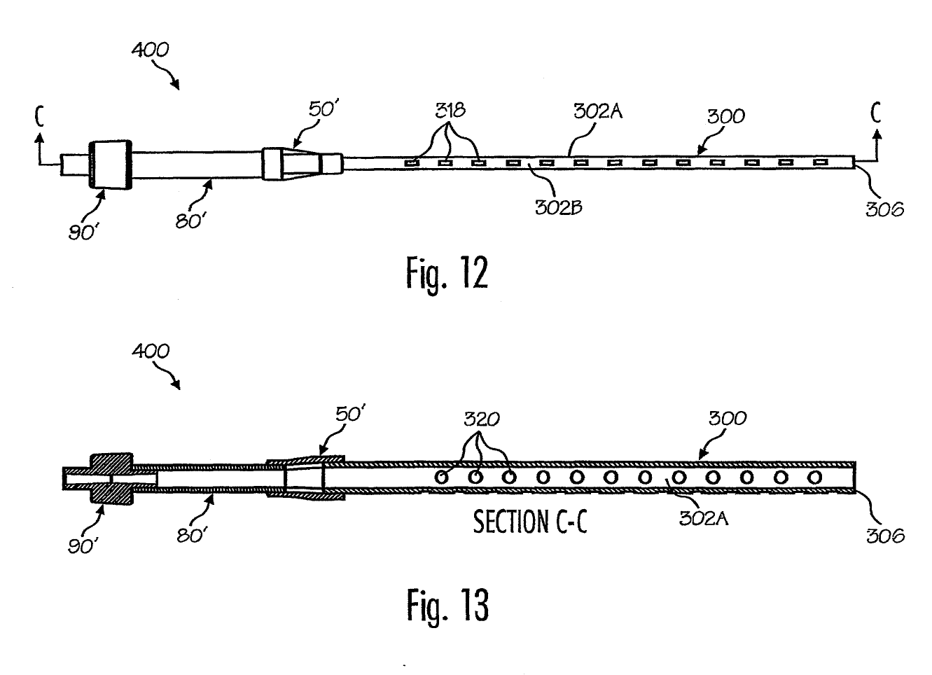

An optional drain system utilizing drain 300 is shown in FIGS. 12-14. The purpose of the system components used with drain 300 is to transport bodily materials to a reservoir (not shown) and any suitable components or system may be used that performs this function. As shown, a collar 50′, having a first section 52′, a second section 54′ and a passage 56′, couples drain 300 to an outflow tube 80′, which has another surface 82′, a first end 84′, a second end 86′ and an internal passage 88′. A connector 90′ attaches to outflow to be 80′ and has a connector 92′, a main body portion 94′, a second connector 96′ and internal passage 98′.

A drain according to the invention can be manufactured to contain four smaller, circular, para-central lumens radially displaced with respect to each other. These additional lumens although they provide a multi-lumen drain by necessity mandate that the overall lumen capable of draining particle debris is reduced by 15% of what the single lumen drain can handle. Such an arrangement provides for stress reduction of the drain.

The drain according to the invention can be manufactured to contain four pie-shaped, para-central lumens radially displaced with respect to each other. The potential benefit that such an arrangement could provide is a wide entrance of 4 mm bit a limited deep recess in the pie-shaped lumen that narrows down to a point. Such a drain would have nearly all of the performance characteristics of the single lumen drain. Such a drain is made with a cross member placed inside the central lumen. Such a cross member would enhance the strength of the drain since it would be less prone to tearing, and it would have multi-lumen capacity.

Thus having described preferred embodiments of the invention, alterations and modifications that do not depart from the spirit of the invention may occur to others. The invention is thus not limited to the preferred embodiments but is instead set forth in the appended claims and legal equivalents thereof.

What is claimed is:

1. A surgical drain comprising:
(a) a longitudinal axis;
(b) an interior lumen;
(c) an outer surface;
(c) an opening for connection to an outflow tube;
(e) a first plurality of openings formed in the outer surface and extending along the longitudinal axis, at least some of the first plurality of openings in fluid communication with the interior lumen; and
(f) a second plurality of openings, each of the second plurality of openings having a different shape and size than the openings of the first plurality of openings.

2. A surgical drain having an elongated, flexible body, two ends, a length and a longitudinal axis, and comprising:
(a) an outer surface;
(b) an interior lumen;
(c) an opening to connect the drain to an outflow tube;
(d) a first plurality of openings formed in the outer surface and in fluid communication with the interior lumen, the first plurality of openings comprising alternating large cross-sectional openings and small cross-sectional openings.

3. The drain of claim 2 wherein the large cross-sectional openings are circular and the small cross-sectional openings are rectangular.

4. The drain of claim 2 wherein the first plurality of openings is formed in the outer surface along the longitudinal axis.

5. The drain of claim 2 that further includes a second plurality of openings comprising alternating large cross-section openings and small cross-sectional openings.

6. The drain of claim 5 wherein the second plurality of openings is formed in the outer surface along the longitudinal axis.

7. The drain of claim 5 wherein the first plurality of openings is formed in the outer surface along the longitudinal axis.

8. The drain of claim 5 wherein the large cross-sectional openings of the second plurality of openings are offset from the large cross-sectional openings of the first plurality of openings.

9. The of claim 5 wherein the small cross-sectional openings of the second plurality of openings are offset from the small cross-sectional openings of the first plurality of openings.

10. The drain of claim 2 wherein the interior lumen extends the length of the drain.

11. The drain of claim 2 wherein the opening is at an end of the drain.

12. The inflow section of claim 11 wherein the end opposite the opening is plugged to prevent bodily material from entering.

13. A surgical drain system comprising the drain of claim 2.

14. The surgical drain system of claim 13 further comprising a reservoir and an outflow to be extending from the drain to the reservoir

15. A surgical drain comprising:
(a) an outer surface, an internal lumen, a generally flat bottom surface and a generally flat top surface;
(b) two side surfaces connecting the top surface and the bottom surface;
(c) circular openings in the top surface; and
(d) non-circular openings in one of the side surfaces.

16. The drain of claim 15 wherein the non-circular openings are rectangular.

17. The drain of claim 15 wherein the circular openings have a diameter of 4 mm.

18. The drain of claim 15 that further includes circular openings on the bottom surface.

19. The drain of claim 15 that includes non-circular openings on both side surfaces.

20. The drain of claim 15 that includes at least one rib in the internal lumen to separate the top surface and the bottom surface.

21. The drain of claim 20 that includes two ribs to separate the top surface and the bottom surface.

22. The drain of claim 2 wherein the lumen has diameter of 6 mm.

23. A surgical drain having an outer surface, an interior lumen, and a channel formed in the outer surface, the channel for impeding the in-growth of bodily tissue therein, and one or more openings formed at least partially in the base of channel, the openings in fluid communication with the interior lumen.

24. The drain of claim 23 wherein there are four channels.

25. The drain of claim 23 wherein the channel is about 0.5 mm deep.

26. The drain of claim 23 wherein the drain has a circular cross-section.

27. The drain of claim 23 wherein the openings are rectangular.

28. The drain of claim 27 that fiber comprises circular openings.

29. The drain of claim 28 wherein the circular openings are formed along the channel.

30. The drain of claim 29 wherein the circular openings alternate with the rectangular openings.
